(19) United States
(12) Patent Application Publication
(10) Pub. No.: US 2018/0015206 A1
(43) Pub. Date: Jan. 18, 2018

(54) BONE REPAIRING MATERIAL AND METHOD FOR PRODUCING THEREOF

(71) Applicant: 3D Global Biotech Inc., New Taipei City (TW)

(72) Inventors: KENG-LIANG OU, New Taipei City (TW); CHIH-HUA YU, New Taipei City (TW); YU-HAO CHAN, New Taipei City (TW); WEI-JEN CHENG, New Taipei City (TW)

(21) Appl. No.: 15/646,576

(22) Filed: Jul. 11, 2017

(30) Foreign Application Priority Data
Jul. 12, 2016 (TW) 105121942

(51) Int. Cl. A61L 27/54; A61L 27/02; A61L 2/00; A61C 8/02; C01F 11/46; A61L 27/36

(52) U.S. Cl. CPC A61L 27/54; C01F 11/466; A61L 27/365; A61L 2/0035; A61L 27/3683; A61C 8/0006; A61L 27/3608; A61L 27/025; A61L 2/0082; A61L 2300/412; A61L 2430/02

(57) ABSTRACT

A bone repairing material comprises a composition including an α-calcium sulfate hemihydrate and an autologous bone powder, wherein the bone repairing material contains 20˜60 weight percent of the autologous bone powder and 40˜80 weight percent of the α-calcium sulfate hemihydrate. The bone repairing material has a particle size in the range of 50˜1,000 μm. A method for producing the bone repairing material comprises the following steps: producing the α-calcium sulfate hemihydrate from a calcium sulfate dihydrate by microwave heating; grinding an autologous bone for generating the autologous bone powder; and, mixing the α-calcium sulfate hemihydrate and the autologous bone powder to form the bone repairing material.

### BACKGROUND OF THE INVENTION

### 1. Field of the Invention

The present invention relates to a bone repairing material and method for producing thereof, and more particularly, to a bone repairing material and method for producing thereof for the field of dental implants.

### 2. Description of the Prior Art

The repair and reconstruction of bone defects have always been an important research direction in orthopedics and plastic surgery. The conventional bone graft materials can be divided into autologous bone graft, allograft and xenotransplantation. The materials of autologous bone graft are obtained from the bones of patients, therefore having the best biocompatibility. The materials of allograft are obtained from the bones of donors, therefore having considerable biocompatibility, but immunosuppressive therapy is needed and the possibility of disease infection is existed. Xenotransplantation obtains the bones from different species, and there are several sources to obtain the bones, but the immune rejection is large, and the possibility of disease transmission is still existed. Artificial bone powder is made by biotechnology synthesis, and the sources of the artificial bone powder are sufficient.

Currently, the autologous bone graft is still the main method of bone defect repair, mainly because the materials of the autogenous bone graft have the best biocompatibility. However, the bone mass of a person that can be used to transplant varies from person to person. Then, the problem of bone deficiency may be induced, the wounds and pain generated when obtaining bone may be increased, and the operation time may be extended. In order to solve and avoid the problems of bone deficiency and immune rejection, the current bone material transplantation is to adopt artificial bone materials for transplantation. Artificial bone materials can be artificial bone powder which is synthesized by the biotechnology, and thus the sources are sufficient to solve the problem of bone deficiency. However, although the artificial bone powder has the ability to guide bone, it does not have the ability to induce bone.

### SUMMARY OF THE INVENTION

Therefore, to solve the above-mentioned problems, the present invention provides a bone repairing material and method for producing thereof, and the bone repairing material and method for producing thereof can solve and avoid the problems of bone deficiency and immune rejection, and also have the ability to induce bone.

The bone repairing material provided by the present invention comprises a composition including an α-calcium sulfate hemihydrate and an autologous bone powder, wherein the bone repairing material contains 20˜60 weight percent of the autologous bone powder and 40˜80 weight percent of the α-calcium sulfate hemihydrate, and the bone repairing material has a particle size in the range of 50˜1,000 μm.

The present invention further provides a method for producing the bone repairing material, comprising the following steps: producing the α-calcium sulfate hemihydrate from a calcium sulfate dihydrate by microwave heating; grinding an autologous bone for generating the autologous bone powder; and mixing the α-calcium sulfate hemihydrate and the autologous bone powder to form the bone repairing material.

The step of producing the α-calcium sulfate hemihydrate from the calcium sulfate dihydrate by microwave heating can selectively comprise the following substeps: placing the calcium sulfate dihydrate and a secondary water into a microwave heating apparatus; heating the microwave heating apparatus to a first predetermined temperature within a second predetermined time; and maintaining the microwave heating apparatus in the first predetermined temperature for a third predetermined time to form a mixed material. Wherein, the first predetermined temperature can be 150˜200° C., and the second predetermined time can be 20 minutes, and the third predetermined time can be 5˜10 minutes. The calcium sulfate dehydrate can be 0.5˜3 g, and the secondary water can be 10˜20 ml, and the output power of the microwave heating apparatus can be 800 watts.

After the step of maintaining the microwave heating apparatus in the first predetermined temperature for the third predetermined time, the method can selectively comprise the following steps: taking out the mixed material placed in the microwave heating apparatus, and processing a cooling filtration by suction filtration and washing the mixed material with anhydrous ethanol; and taking out the mixed material through the cooling filtration and washed with the anhydrous ethanol to be baked by an oven to obtain the α-calcium sulfate hemihydrate.

The step of grinding the autologous bone for generating the autologous bone powder can selectively comprise the following substeps: cleaning the autologous bone; grinding and shaking the autologous bone into the autologous bone powder; and sterilizing and drying the grinded and shaken autologous bone powder.

The step of mixing the α-calcium sulfate hemihydrate and the autologous bone powder to form the bone repairing material can selectively comprise the following substeps: taking the autologous bone powder and the α-calcium sulfate hemihydrate into a stirring device and stirring for a first predetermined time, wherein the autologous bone powder is accounted for 20˜60 weight percent of the bone repairing material and the α-calcium sulfate hemihydrate is accounted for 40˜80 weight percent of the bone repairing material; and processing a sterilization treatment to the stirred bone repairing material. Wherein, the first predetermined time can be 10 minutes.

In conclusion, the present invention provides a bone repairing material and method for producing thereof. The bone repairing material provided by the present invention is produced through combining the autologous bone powder with the α-calcium sulfate hemihydrate, and the method for producing the bone repairing material is: producing the α-calcium sulfate hemihydrate from the calcium sulfate dihydrate by microwave heating; grinding the autologous bone for generating the autologous bone powder; and mixing the α-calcium sulfate hemihydrate and the autologous bone powder to form the bone repairing material. The bone repairing material provided by the present invention is produced through combining the autologous bone powder with the α-calcium sulfate hemihydrate; therefore, the problems of bone deficiency and immune rejection can be avoided, and the present invention can also be applied to the case of wide range of bone defect.

The advantages and spirits of the invention may be understood by the following recitations together with the appended drawings.

### BRIEF DESCRIPTION OF THE APPENDED DRAWINGS

Some of the embodiments will be described in detail, with reference to the following figures, wherein like designations denote like members, wherein:

FIG. 1 shows a flow chart of the method for producing the bone repairing material in an embodiment of the present invention.

FIG. 2 shows a flow chart of the method for producing the bone repairing material in an embodiment of the present invention.

FIG. 3 shows a flow chart of the method for producing the α-calcium sulfate hemihydrate in an embodiment of the present invention.

FIG. 4 shows a flow chart of the method for producing the α-calcium sulfate hemihydrate in another embodiment of the present invention.

FIG. 5A and FIG. 5B show a pattern diagram of the osteocyte cultured on the surface of the α-calcium sulfate hemihydrate.

FIG. 6 shows a result diagram of the method for the cytotoxicity test of the α-calcium sulfate hemihydrate.

FIG. 7 shows a flow chart of the method for producing the autologous bone powder in an embodiment of the present invention.

FIG. 8 shows a flow chart of the method for producing the autologous tooth powder in an embodiment of the present invention.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

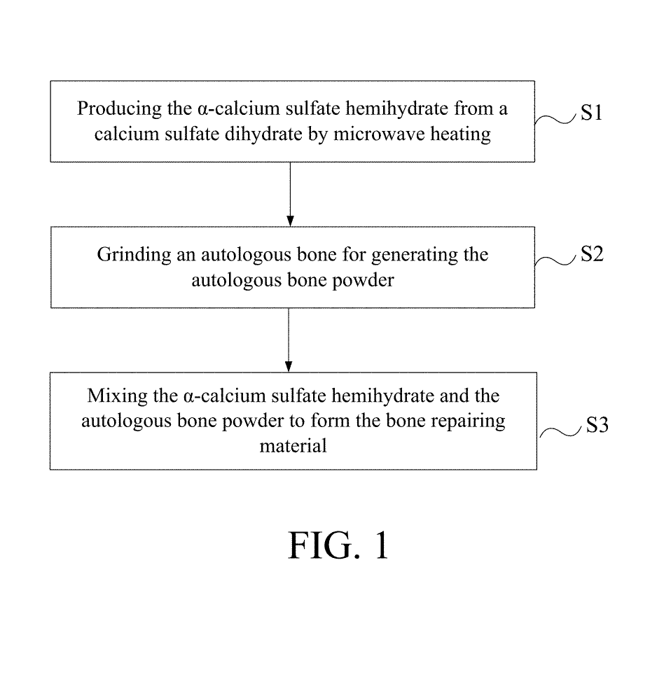

Please refer to FIG. 1. FIG. 1 shows a flow chart of the method for producing the bone repairing material in an embodiment of the present invention, wherein the method comprises the following steps: (S1) producing the α-calcium sulfate hemihydrate from a calcium sulfate dihydrate by microwave heating; (S2) grinding an autologous bone for generating the autologous bone powder; and (S3) mixing the α-calcium sulfate hemihydrate and the autologous bone powder to form the bone repairing material. In an embodiment of the present invention, the bone repairing material provided by the method of FIG. 1 comprises a composition including the α-calcium sulfate hemihydrate and the autologous bone powder, wherein the bone repairing material contains 20˜60 weight percent of the autologous bone powder and 40˜80 weight percent of the α-calcium sulfate hemihydrate, and the bone repairing material has a particle size in the range of 50˜1,000 μm.

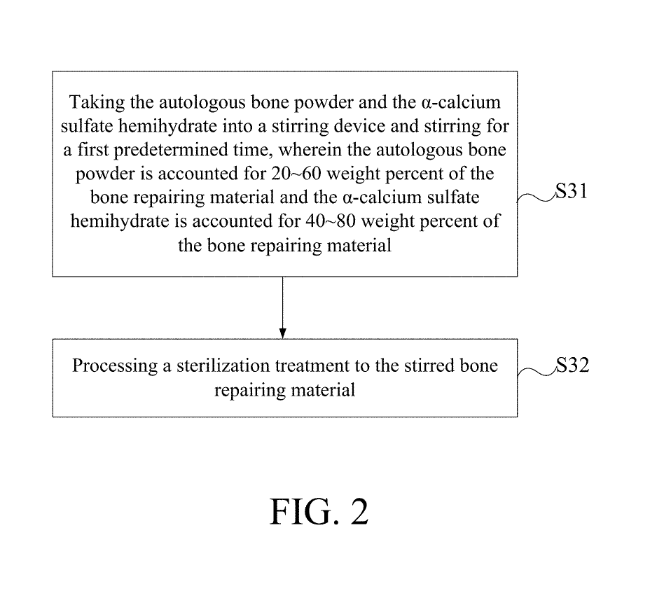

Please refer to FIG. 2. FIG. 2 shows a flow chart of the method for producing the bone repairing material in an embodiment of the present invention. In an embodiment of the present invention, as shown in FIG. 2, the step of (S3) mixing the α-calcium sulfate hemihydrate and the autologous bone powder to form the bone repairing material further comprises the following substeps: (S31) taking the autologous bone powder and the α-calcium sulfate hemihydrate into a stirring device and stirring for a first predetermined time, wherein the autologous bone powder is accounted for 20˜60 weight percent of the bone repairing material and the α-calcium sulfate hemihydrate is accounted for 40˜80 weight percent of the bone repairing material; and then (S32) processing a sterilization treatment to the stirred bone repairing material.

In an embodiment of the present invention, in the step of (S31) taking the autologous bone powder and the α-calcium sulfate hemihydrate into the stirring device and stirring for the first predetermined time, wherein the autologous bone powder is accounted for 20˜60 weight percent of the bone repairing material and the α-calcium sulfate hemihydrate is accounted for 40˜80 weight percent of the bone repairing material; the first predetermined time can be, but not limited to, 10 minutes, and the first predetermined time can be adjusted according to users' demand time in the practical application. In the embodiment of the present invention, the stirring device can be, but not limited to, mixing bowl and spoon, and the stirring device can also be any devices that can stir the autologous bone powder and the α-calcium sulfate hemihydrate evenly in the practical application. Besides, in an embodiment of the present invention, a grinding device can also be used to grind the autologous bone powder and the α-calcium sulfate hemihydrate taken out of the scale for generating the bone repairing material, wherein the autologous bone powder is accounted for 20˜60 weight percent of the bone repairing material and the α-calcium sulfate hemihydrate is accounted for 40˜80 weight percent of the bone repairing material.

In an embodiment of the present invention, in the step of (S32) processing the sterilization treatment to the stirred bone repairing material, the sterilization treatment can be, but not limited to, pouring the bone repairing material into a packaging bottle for packaging and sending to Gamma for sterilization, and the sterilization treatment can be adjusted according to users' needs in the practical application.

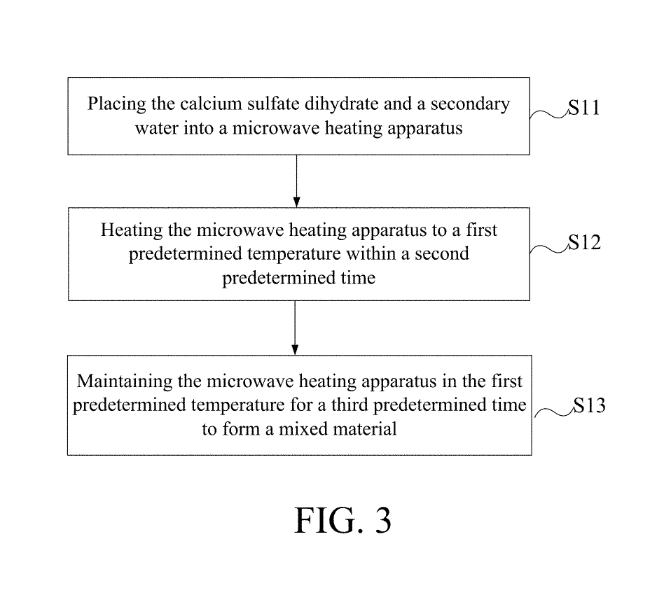

Please refer to FIG. 3. FIG. 3 shows a flow chart of the method for producing the α-calcium sulfate hemihydrate in an embodiment of the present invention. In an embodiment of the present invention, as shown in FIG. 3, the method for producing the α-calcium sulfate hemihydrate is (S1) producing the α-calcium sulfate hemihydrate from a calcium sulfate dihydrate by microwave heating, and the method can comprise the following substeps: (S11) placing the calcium sulfate dihydrate and a secondary water into a microwave heating apparatus; (S12) heating the microwave heating apparatus to a first predetermined temperature within a second predetermined time; and (S13) maintaining the microwave heating apparatus in the first predetermined temperature for a third predetermined time to form a mixed material.

In an embodiment of the present invention, in the step of (S11) placing the calcium sulfate dihydrate and the secondary water into the microwave heating apparatus, the calcium sulfate dehydrate can be 0.5˜3 g, and the secondary water can be 10˜20 ml, and the output power of the microwave heating apparatus can be set to, but not limited to, 800 watts, and the output power can be adjusted according to users' needs in the practical application.

In an embodiment of the present invention, in the step of (S12) heating the microwave heating apparatus to the first predetermined temperature within the second predetermined time, the first predetermined temperature can be, but not limited to, 150˜200° C., and the second predetermined time can be, but not limited to, 20 minutes, and heating parameter and rate can be adjusted according to users' needs in the practical application.

In an embodiment of the present invention, in the step of (S13) maintaining the microwave heating apparatus in the first predetermined temperature for the third predetermined time to form the mixed material, the first predetermined temperature can be, but not limited to, 150˜200° C., and the third predetermined time can be, but not limited to, 5˜10 minutes, and holding temperature parameter can be adjusted according to users' needs in the practical application.

In an embodiment of the present invention, after the step of (S13) maintaining the microwave heating apparatus in the first predetermined temperature for the third predetermined time to form the mixed material, the method further comprises the following steps: (S131) taking out the mixed material placed in the microwave heating apparatus, and processing a cooling filtration by suction filtration and washing the mixed material with anhydrous ethanol; and (S132) taking out the mixed material through the cooling filtration and washed with the anhydrous ethanol to be baked by an oven to obtain the α-calcium sulfate hemihydrate.

In an embodiment of the present invention, the step of (S131) taking out the mixed material placed in the microwave heating apparatus, and processing the cooling filtration by suction filtration and washing the mixed material with anhydrous ethanol is taking out the mixed material after heating and holding temperature from the microwave heating apparatus and importing to place on a filter paper of suction filtration device, and then processing the cooling filtration by suction filtration and washing the mixed material with anhydrous ethanol simultaneously about 2˜3 minutes.

In an embodiment of the present invention, in the step of (S132) taking out the mixed material through the cooling filtration and washed with the anhydrous ethanol to be baked by an oven to obtain the α-calcium sulfate hemihydrate, the mixed material through the cooling filtration and washed with the anhydrous ethanol is baked at, but not limited to, 60 degrees, and the baking temperature and time can be adjusted according to users' needs in the practical application.

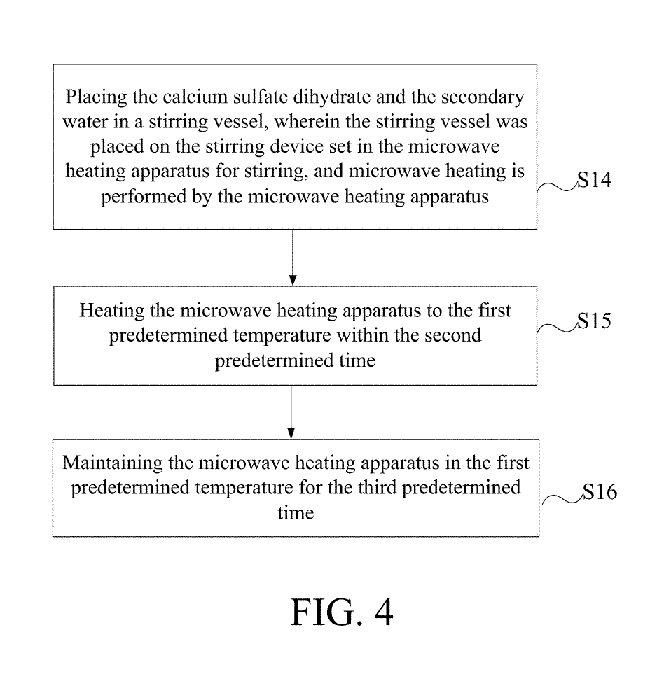

FIG. 4 shows a flow chart of the method for producing the α-calcium sulfate hemihydrate in another embodiment of the present invention. In an embodiment of the present invention, as shown in FIG. 4, the method for producing the α-calcium sulfate hemihydrate is (S1) producing the α-calcium sulfate hemihydrate from the calcium sulfate dihydrate by microwave heating, and the method can comprise the following substeps: (S14) placing the calcium sulfate dihydrate and the secondary water in a stirring vessel, wherein the stirring vessel was placed on the stirring device set in the microwave heating apparatus for stirring, and microwave heating is performed by the microwave heating apparatus; (S15) heating the microwave heating apparatus to the first predetermined temperature within the second predetermined time; and (S16) maintaining the microwave heating apparatus in the first predetermined temperature for the third predetermined time to hold the temperature.

In an embodiment of the present invention, in the step of (S14) placing the calcium sulfate dihydrate and the secondary water in the stirring vessel, wherein the stirring vessel was placed on the stirring device set in the microwave heating apparatus for stirring, and microwave heating is performed by the microwave heating apparatus, the calcium sulfate dehydrate can be 0.5˜3 g, and the secondary water can be 10˜20 ml, and the output power of the microwave heating apparatus can be set to, but not limited to, 800 watts, and the output power can be adjusted according to users' needs in the practical application. Besides, in the embodiment of the present invention, the mentioned stirring of the stirring device can be performed through placing the calcium sulfate dihydrate, the secondary water, and the stirrer in the stirring vessel by means of magnetic force and mechanical simultaneous, and then placing the stirring vessel on an agitator set in the microwave heating apparatus to drive the stirrer to rotate at a constant rotational speed by a magnetic rotor in the agitator, and microwave heating is performed by the microwave heating apparatus, wherein the stirrer can be a magnet.

In an embodiment of the present invention, in the step of (S15) heating the microwave heating apparatus to the first predetermined temperature within the second predetermined time, the first predetermined temperature can be, but not limited to, 150˜200° C., and the second predetermined time can be, but not limited to, 20 minutes, and heating parameter and rate can be adjusted according to users' needs in the practical application.

In an embodiment of the present invention, in the step of (S16) maintaining the microwave heating apparatus in the first predetermined temperature for the third predetermined time to hold the temperature, the first predetermined temperature can be, but not limited to, 150˜200° C., and the third predetermined time can be, but not limited to, 5˜10 minutes, and holding temperature parameter can be adjusted according to users' needs in the practical application.

In an embodiment of the present invention, after the step of (S16) maintaining the microwave heating apparatus in the first predetermined temperature for the third predetermined time to hold the temperature, the method further comprises the following steps: (S161) taking out the mixed material placed in the microwave heating apparatus, and processing the cooling filtration by suction filtration and washing the mixed material with anhydrous ethanol; and (S162) taking out the mixed material through the cooling filtration and washed with the anhydrous ethanol to be baked by the oven to obtain the α-calcium sulfate hemihydrate.

In an embodiment of the present invention, the step of (S161) taking out the mixed material placed in the microwave heating apparatus, and processing the cooling filtration by suction filtration and washing the mixed material with anhydrous ethanol is taking out the mixed material after heating and holding temperature from the microwave heating apparatus and importing to place on the filter paper of suction filtration device, and then processing the cooling filtration by suction filtration and washing the mixed material with anhydrous ethanol simultaneously about 2˜3 minutes. While in the step of (S162) taking out the mixed material through the cooling filtration and washed with the anhydrous ethanol to be baked by an oven to obtain the α-calcium sulfate hemihydrate, the mixed material through the cooling filtration and washed with the anhydrous ethanol is baked at, but not limited to, 60 degrees, and the baking temperature and time can be adjusted according to users' needs in the practical application.

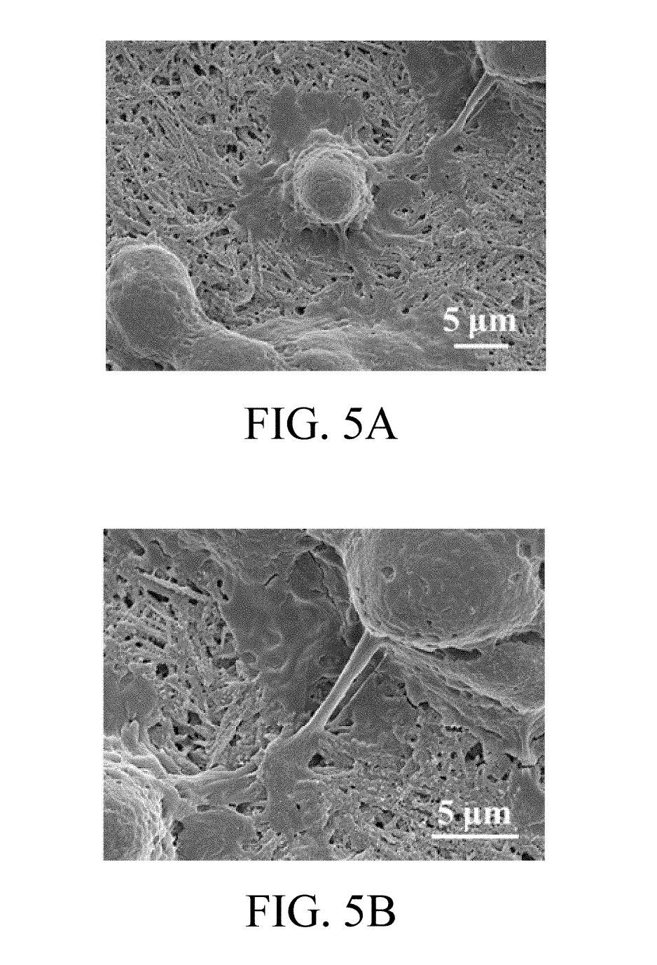

Next, please refer to FIG. 5A and FIG. 5B. FIG. 5A and FIG. 5B show a pattern diagram of the osteocyte cultured on the surface of the α-calcium sulfate hemihydrate. In FIG. 5A and FIG. 5B, show the pattern diagram of the osteocyte cultured on the surface of the α-calcium sulfate hemihydrate to observe the cell attachment pattern, wherein the α-calcium sulfate hemihydrate in the present invention goes through stirring, microwave heating, cooling filtering, anhydrous ethanol filtering and baking. As shown in FIG. 5A and FIG. 5B, the osteocyte can be attached normally to the surface of the α-calcium sulfate hemihydrate.

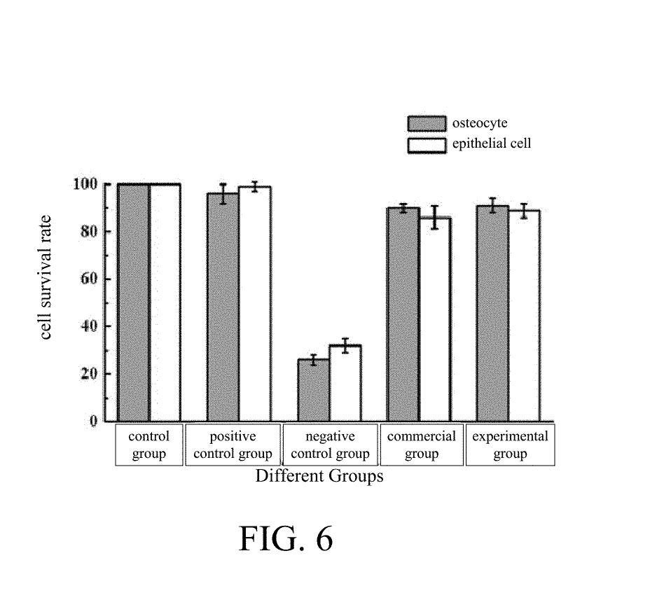

Besides, please refer to FIG. 6. FIG. 6 shows a result diagram of the method for the cytotoxicity test of the α-calcium sulfate hemihydrate, wherein FIG. 6 shows the result data diagram of the osteocyte and the epithelial cell vitro cultured in the cytotoxicity test of the α-calcium sulfate hemihydrate extract. Wherein the horizontal axis represents the control group, the positive control group (HDPE, high-density polyethylene), the negative control group (ZDBC, zincdibutyl dithiocarbamate), the commercial group (commercially available α-calcium sulfate hemihydrate), the experimental group (the α-calcium sulfate hemihydrate produced through stirring, microwave heating, cooling filtering, anhydrous ethanol filtering and baking), and the vertical axis represents cell survival rate. It can be learned that the α-calcium sulfate hemihydrate has no cytotoxicity from the result data diagram of FIG. 6.

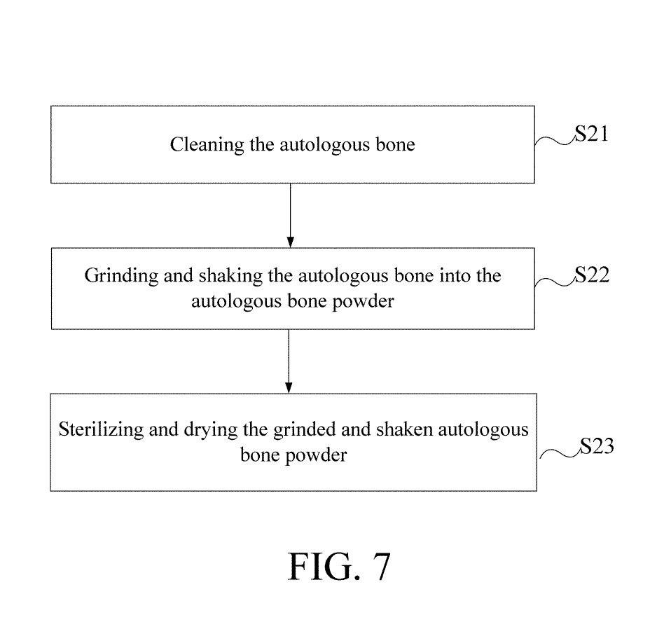

Please refer to FIG. 7. FIG. 7 shows a flow chart of the method for producing the autologous bone powder in an embodiment of the present invention. In an embodiment of the present invention, as shown in FIG. 7, the method for producing the autologous bone powder is (S2) grinding the autologous bone for generating the autologous bone powder, and the method further comprises the following substeps: (S21) cleaning the autologous bone; (S22) grinding and shaking the autologous bone into the autologous bone powder; and (S23) sterilizing and drying the grinded and shaken autologous bone powder.

In an embodiment of the present invention, the step of (S21) cleaning the autologous bone can comprise the following substeps: (S211) washing the autologous bone by water; (S212) wiping the autologous bone by hydrogen peroxide; and (S214) the autogenous bone can be blown dry with compressed air and kept dry after the cleaning is completed.

In an embodiment of the present invention, the step of (S212) wiping the autologous bone by hydrogen peroxide, in the practical application, the method can be, but not limited to, soaking the autologous bone in hydrogen peroxide, and then taking out the autologous bone for hydrogen peroxide to volatilize after a certain time.

In an embodiment of the present invention, after the step of (S214) the autogenous bone can be blown dry with compressed air and kept dry after the cleaning is completed, the method can also comprise the following steps: (S215) placing the cleaned autogenous bone in the box and taken out when the grinding step is processed.

In an embodiment of the present invention, the step of (S22) grinding and shaking the autologous bone into the autologous bone powder can comprise the following substeps: (S221) placing the autogenous bone on an actuating device for grinding and shaking; and (S222) grinding and shaking the autogenous bone respectively.

In an embodiment of the present invention, in the step of (S222) grinding and shaking the autogenous bone respectively, the time of grinding and shaking and the number of times of grinding and shaking can be adjusted according to the needs of grinding the autogenous bone to obtain a grinded evenly autogenous bone powder.

In an embodiment of the present invention, in the step of (S23) sterilizing and drying the grinded and shaken autologous bone powder, the method can comprise the sterilizing step and the drying step.

In an embodiment of the present invention, the sterilizing step can comprise the following substeps: (S231) pouring the grinded and shaken autologous bone powder into a box; (S232) adding the disinfectant to the box that is placed in the grinded autologous bone powder by a removing device so that the disinfectant is flooded through the autologous bone powder; (S233) taking out the disinfectant by the removing device; (S234) completing the sterilization of the autologous bone powder. Wherein the sterilizing step is not limited to the above-mentioned description, and the step of adding and taking out the disinfectant can also be repeated according to the needs in the practical application.

In an embodiment of the present invention, in the step of (S231) pouring the grinded and shaken autologous bone powder into the box, the box can be a sterile preservation box.

In an embodiment of the present invention, the drying step can comprise the following substeps: (S235) placing the box that is placed in the sterilized autologous bone powder in a drying device to be dried for a predetermined time; (S236) placing the autologous bone powder in a cold storage device to preserve the autologous bone powder after the drying device is dried.

In an embodiment of the present invention, in the step of (S235) placing the box that is placed in the sterilized autologous bone powder in the drying device to be dried for the predetermined time, the predetermined time is, but not limited to, 20˜30 minutes, and the time and number of times of drying can be adjusted according to the autologous bone powder amount in the practical application.

In an embodiment of the present invention, in the step of (S236) placing the autologous bone powder in the cold storage device to preserve the autologous bone powder after the drying device is dried, the cold storage device can be a refrigerator.

In conclusion, the present invention provides a bone repairing material and method for producing thereof. The bone repairing material provided by the present invention is produced through combining the autologous bone powder with the α-calcium sulfate hemihydrate, and the method for producing the bone repairing material is: producing the α-calcium sulfate hemihydrate from the calcium sulfate dihydrate by microwave heating; grinding the autologous bone for generating the autologous bone powder; and mixing the α-calcium sulfate hemihydrate and the autologous bone powder to form the bone repairing material. The bone repairing material provided by the present invention is produced through combining the autologous bone powder with the α-calcium sulfate hemihydrate; therefore, the problems of bone deficiency and immune rejection can be avoided, and the present invention can also be applied to the case of wide range of bone defect.

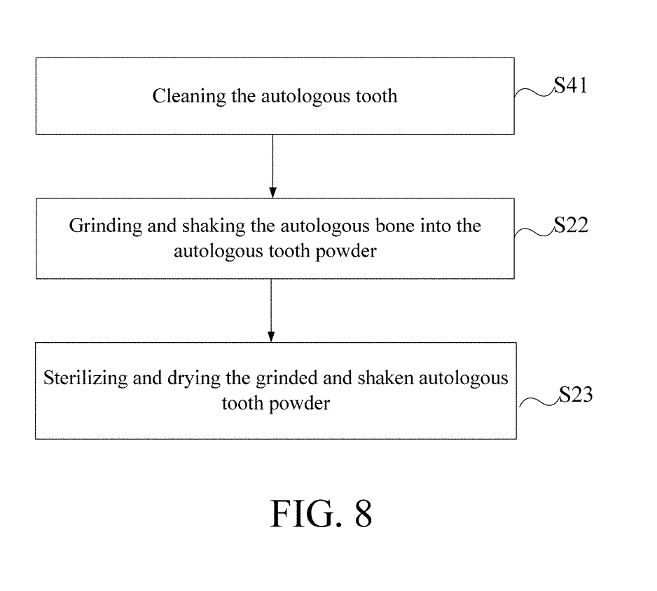

Please refer to FIG. 8. FIG. 8 shows a flow chart of the method for producing the autologous tooth powder in an embodiment of the present invention. In an embodiment of the present invention, the autologous bone powder provided by the method in FIG. 8 can be an autologous tooth powder, and the α-calcium sulfate hemihydrate is mixed with the autologous tooth powder to form the bone repair material which can be applied to the field of implants for dental restoration. The method for producing the α-calcium sulfate hemihydrate in the embodiment of the present invention has been described in the previous paragraph, therefore details will not repeated herein again. In the embodiment of the present invention, the method for producing the autologous tooth powder is (S4) grinding an autologous tooth for generating the autologous tooth powder, wherein the method can comprise the following substeps: (S41) cleaning the autologous tooth; (S42) grinding and shaking the autologous tooth into the autologous tooth powder; and (S23) sterilizing and drying the grinded and shaken autologous tooth powder.

In an embodiment of the present invention, the step of (S41) cleaning the autologous tooth can comprise the following substeps: (S411) washing the autologous tooth by water; (S412) wiping the autologous tooth by hydrogen peroxide; (S413) removing non-dentine by high speed round bur; and (S414) the autogenous tooth can be blown dry with compressed air and kept dry after the cleaning is completed.

In an embodiment of the present invention, the step of (S412) wiping the autologous tooth by hydrogen peroxide, in the practical application, the method can be, but not limited to, soaking the autologous tooth in hydrogen peroxide, and then taking out the autologous tooth for hydrogen peroxide to volatilize after a certain time.

In an embodiment of the present invention, in the step of (S413) removing non-dentine by high speed round bur, the non-dentine can be prosthesis material, denture or calculus.

In an embodiment of the present invention, the step of (S42) grinding and shaking the autologous tooth into the autologous tooth powder can comprise the following substeps: (S421) placing the autogenous tooth on an actuating device for grinding and shaking; and (S422) grinding and shaking the autogenous tooth respectively.

In an embodiment of the present invention, in the step of (S422) grinding and shaking the autogenous tooth respectively, the time of grinding can be, but not limited to, 3 seconds, and the time of shaking can be, but not limited to, 20 seconds. In the practical application, the time of grinding and shaking and the number of times of grinding and shaking can be adjusted according to the needs of grinding the autogenous tooth to obtain a grinded evenly autogenous tooth powder.

In an embodiment of the present invention, in the step of (S43) sterilizing and drying the grinded and shaken autologous tooth powder, the method can comprise the sterilizing step and the drying step.

In an embodiment of the present invention, the sterilizing step can comprise the following substeps: (S431) pouring the grinded and shaken autologous tooth powder into a box; (S432) adding the disinfectant to the box that is placed in the grinded autologous tooth powder by a removing device so that the disinfectant is flooded through the autologous tooth powder; (S433) taking out the disinfectant by the removing device; (S434) completing the sterilization of the autologous tooth powder. In an embodiment of the present invention, in the step of (S431) pouring the grinded and shaken autologous tooth powder into the box, the box can be a sterile preservation box.

Wherein the step of sterilizing is not limited to the above-mentioned, the step of sterilizing mentioned above can comprise the step of adding and taking out the disinfectant twice, wherein the steps are described below: (S4341) adding the disinfectant to the box that is placed in the grinded autologous tooth powder by the removing device so that the disinfectant is flooded through the autologous tooth powder and soaked for a predetermined time; (S4342) taking out the disinfectant by the removing device after being soaked for a predetermined time; (S4343) adding the disinfectant to the box that is placed in the autologous tooth powder after the first sterilization by the removing device again so that the disinfectant is flooded through the autologous tooth powder and soaked for another predetermined time; (S4344) taking out the disinfectant by the removing device again; (S4345) completing the sterilization of the autologous tooth powder.

In an embodiment of the present invention, in the step of (S4341) adding the disinfectant to the box that is placed in the grinded autologous tooth powder by the removing device so that the disinfectant is flooded through the autologous tooth powder and soaked for the predetermined time, the predetermined time is, but not limited to, 10 minutes, and the time of soaking can be adjusted according to the sterilization needs in the practical application.

In an embodiment of the present invention, in the step of (S4343) adding the disinfectant to the box that is placed in the autologous tooth powder after the first sterilization by the removing device again so that the disinfectant is flooded through the autologous tooth powder and soaked for another predetermined time, the predetermined time is, but not limited to, 3 minutes, and the time of soaking can be adjusted according to the sterilization needs in the practical application.

In an embodiment of the present invention, the drying step can comprise the following substeps: (S435) placing the box that is placed in the sterilized autologous tooth powder in a drying device to be dried for a predetermined time; (S436) placing the autologous tooth powder in a cold storage device to preserve the autologous tooth powder after the drying device is dried.

In an embodiment of the present invention, in the step of (S435) placing the box that is placed in the sterilized autologous tooth powder in the drying device to be dried for the predetermined time, the predetermined time is, but not limited to, 20˜30 minutes, and the time and number of times of drying can be adjusted according to the autologous tooth powder amount in the practical application.

In an embodiment of the present invention, in the step of (S436) placing the autologous tooth powder in the cold storage device to preserve the autologous tooth powder after the drying device is dried, the cold storage device can be a refrigerator.

In conclusion, the present invention provides a bone repairing material and method for producing thereof, wherein the present invention can be applied to the field of implants. The bone repairing material provided by the present invention is produced through combining the autologous bone powder with the α-calcium sulfate hemihydrate, and the method for producing the bone repairing material is: producing the α-calcium sulfate hemihydrate from the calcium sulfate dihydrate by microwave heating; grinding the autologous bone for generating the autologous bone powder; and mixing the α-calcium sulfate hemihydrate and the autologous bone powder to form the bone repairing material. The bone repairing material provided by the present invention is produced through combining the autologous bone powder with the α-calcium sulfate hemihydrate; therefore, the problems of bone deficiency and immune rejection can be avoided, and the present invention can also be applied to the case of wide range of bone defect.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A bone repairing material comprises a composition including an α-calcium sulfate hemihydrate and an autologous bone powder, wherein the bone repairing material contains 20˜60 weight percent of the autologous bone powder and 40˜80 weight percent of the α-calcium sulfate hemihydrate, and the bone repairing material has a particle size in the range of 50˜1,000 μm.

2. A method for producing the bone repairing material of claim 1, comprising the following steps:
producing the α-calcium sulfate hemihydrate from a calcium sulfate dihydrate by microwave heating;
grinding an autologous bone for generating the autologous bone powder; and
mixing the α-calcium sulfate hemihydrate and the autologous bone powder to form the bone repairing material.

3. The method for producing the bone repairing material of claim 2, wherein the step of mixing the α-calcium sulfate hemihydrate and the autologous bone powder to form the bone repairing material further comprises the following substeps:
taking the autologous bone powder and the α-calcium sulfate hemihydrate into a stirring device and stirring for a first predetermined time, wherein the autologous bone powder is accounted for 20˜60 weight percent of the bone repairing material and the α-calcium sulfate hemihydrate is accounted for 40˜80 weight percent of the bone repairing material; and
processing a sterilization treatment to the stirred bone repairing material.

4. The method for producing the bone repairing material of claim 3, wherein the first predetermined time is 10 minutes.

5. The method for producing the bone repairing material of claim 2, wherein the step of producing the α-calcium sulfate hemihydrate from the calcium sulfate dihydrate by microwave heating further comprises the following substeps:
placing the calcium sulfate dihydrate and a secondary water into a microwave heating apparatus;
heating the microwave heating apparatus to a first predetermined temperature within a second predetermined time; and
maintaining the microwave heating apparatus in the first predetermined temperature for a third predetermined time to form a mixed material.

6. The method for producing the bone repairing material of claim 5, wherein the first predetermined temperature is 150˜200° C.

7. The method for producing the bone repairing material of claim 5, wherein the second predetermined time is 20 minutes, and the third predetermined time is 5˜10 minutes.

8. The method for producing the bone repairing material of claim 5, wherein after the step of maintaining the microwave heating apparatus in the first predetermined temperature for the third predetermined time, the method further comprises the following steps:
taking out the mixed material placed in the microwave heating apparatus, and processing a cooling filtration by suction filtration and washing the material with anhydrous ethanol; and
taking out the mixed material through the cooling filtration and washed with the anhydrous ethanol to be baked by an oven to obtain the α-calcium sulfate hemihydrate.

9. The method for producing the bone repairing material of claim 5, wherein the calcium sulfate dehydrate is 0.5˜3 g, and the secondary water is 10˜20 ml, and the output power of the microwave heating apparatus is 800 watts.

10. The method for producing the bone repairing material of claim 2, wherein the step of grinding the autologous bone for generating the autologous bone powder further comprises the following substeps:
cleaning the autologous bone;
grinding and shaking the autologous bone into the autologous bone powder; and
sterilizing and drying the grinded and shaken autologous bone powder.
